(19) United States
(12) Patent Application Publication
(10) Pub. No.: US 2012/0029567 A1
(43) Pub. Date: Feb. 2, 2012

(54) ANCHORING MECHANISM

(75) Inventors: Aleksandr G. Zolotov, Collierville, TN (US); Carlos E. Gil, Collierville, TN (US)

(73) Assignee: WARSAW ORTHOPEDIC, INC., Warsaw, IN (US)

(21) Appl. No.: 12/847,524

(22) Filed: Jul. 30, 2010

(51) Int. Cl. A61B 17/70; A61B 17/86

(52) U.S. Cl. 606/264; 606/305

(57) ABSTRACT

A system for anchoring at least a portion of material to a vertebral body is disclosed. The anchoring system comprises a base configured to affix to the vertebral body, configured to receive the at least a portion of material, and configured to receive a fastener so as to apply pressure to the at least a portion of material so as to anchor the at least a portion of material to the base. Another system further comprises an anchoring mechanism configured to engage with the base and configured to anchor the at least a portion of material, wherein the anchoring mechanism comprises at least one elastic element configured to engage with a fastener and configured to apply pressure to the at least a portion of material so as to anchor the at least a portion of material to the base.

The present disclosure is related to commonly owned and co-pending U.S. application Ser. No. ______ (having Attorney Docket No. P37336.00), which has a filing date that is the same as the present disclosure, and which is hereby incorporated herein by reference in its entirety.

### FIELD OF INVENTION

The present invention is directed to systems or mechanisms for affixing material to bone.

### BACKGROUND

The present disclosure relates to mechanisms for affixing material to bone, and more particularly, systems for affixing at least a portion of material to a vertebral body.

### SUMMARY OF THE INVENTION

A system for anchoring at least a portion of material to a vertebral body is disclosed. The anchoring system comprises a base configured to affix to the vertebral body and configured to receive the at least a portion of material, a fastener configured to affix the base to the vertebral body, and an anchoring mechanism configured to engage with the base and configured to anchor the at least a portion of material, wherein the anchoring mechanism comprises at least one elastic element configured to engage with the fastener and configured to apply pressure to the at least a portion of material so as to anchor the at least a portion of material to the base.

Another system for anchoring at least a portion of material to a vertebral body is disclosed. The anchoring system comprises a base configured to affix to the vertebral body, configured to receive the at least a portion of material, and configured to receive a fastener so as to apply pressure to the at least a portion of material so as to anchor the at least a portion of material to the base.

Another system for anchoring at least a portion of material to a vertebral body is disclosed. The anchoring comprises a base configured to affix to the vertebral body and configured to receive the at least a portion of material, a fastener comprising a screw and a nut—the fastener configured to affix the base to the vertebral body, and an anchoring mechanism configured to engage with the base and configured to anchor the at least a portion of material, wherein the anchoring mechanism comprises at least one elastic element configured to engage with the nut and configured to apply pressure to the at least a portion of material so as to anchor the at least a portion of material to the base.

Additional aspects and features of the present disclosure will be apparent from the detailed description and claims as set forth below.

### BRIEF DESCRIPTION OF THE DRAWINGS

FIG. 1 is a schematic, cross-sectional view of two adjacent vertebral bodies;

FIG. 2 is a schematic, side view of the vertebral bodies of FIG. 1;

FIG. 3 is an isometric view of an anchoring system;

FIG. 4 is an isometric view of a base and an anchoring mechanism of the anchoring system of FIG. 3;

FIG. 5 is an isometric view of an anchoring mechanism of the anchoring system of FIG. 3 in cooperation with a fastener;

FIG. 6 is another isometric view of an anchoring mechanism of the anchoring system of FIG. 3 in cooperation with a fastener;

FIG. 7 is an isometric view of another anchoring system;

FIG. 8 is a top isometric view of a base of the anchoring system of FIG. 7;

FIG. 9 is a bottom isometric view of a base of the anchoring system of FIG. 7;

FIG. 10 is an isometric view of a base of the anchoring system of FIG. 7 in cooperation with at least a portion of material;

FIG. 11 is an isometric view of a base and a fastener of the anchoring system of FIG. 7 in cooperation with at least a portion of material;

FIG. 12 is a schematic, side view of vertebral bodies with another anchoring system;

FIG. 13 is an isometric view of another anchoring system;

FIG. 14 is a top isometric view of a base of the anchoring system of FIG. 13;

FIG. 15 is an isometric view of a base of the anchoring system of FIG. 13 in cooperation with at least a portion of material;

FIG. 16 is an isometric view of another anchoring system;

FIG. 17 is a top isometric view of a base of an anchoring system of FIG. 16;

FIG. 18 is a bottom isometric view of a base of the anchoring system of FIG. 16;

FIG. 19 is an isometric view of a fastener and locking element of the anchoring system of FIG. 16;

FIG. 20 is an isometric view of another anchoring system;

FIG. 21 is an isometric view of a fastener of the anchoring system of FIG. 20;

FIG. 22 is a top isometric view of a base 420 of the anchoring system of FIG. 20 in cooperation with a fastener;

FIG. 23 is a bottom isometric view of a base and a fastener of the anchoring system of FIG. 20;

FIG. 24 is an isometric view of a fastener and locking element 460 of the anchoring system of FIG. 20;

FIG. 25 is an isometric view of another anchoring system 600;

FIG. 26 is an isometric view of a fastener of the anchoring system of FIG. 24;

FIG. 27 is an isometric view of a base and a fastener of the anchoring system of FIG. 25;

FIG. 28 is an isometric view of a base and a anchoring mechanism of the anchoring system of FIG. 25;

FIG. 29 is an isometric view of a base and an anchoring mechanism of the anchoring system of FIG. 25 in cooperation with a fastener and at least a portion of material;

FIG. 30 is a cross-sectional view of a base and an anchoring mechanism of the anchoring system of FIG. 25 in cooperation with a fastener, at least a portion of material and a locking component;

FIG. 31 is an isometric view of an anchoring mechanism of the anchoring system of FIG. 25;

FIG. 32 is a top view of the anchoring mechanism of FIG. 31;

FIG. 33 is an isometric view of another anchoring system; and

FIG. 34 is an isometric view of the anchoring system of FIG. 33 without a base.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

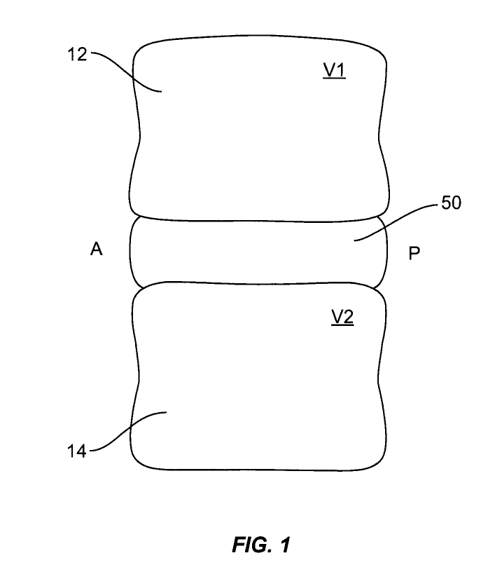

FIG. 1 shows a schematic, cross-sectional view of two adjacent vertebral bodies V1 and V2 with an intervertebral disc 50 situated in its natural location between the two vertebral bodies V1 and V2. As shown in FIG. 1, vertebral body V1 represents a superior vertebral body and V2 represents an inferior vertebral body. Reference marker A represents an anterior side of the vertebral bodies V1 and V2, whereas reference marker P represents a posterior side of the vertebral bodies V1 and V2. As shown in FIG. 1, superior vertebral body V1 has a lateral surface 12 and inferior vertebral body V2 has a lateral surface 14.

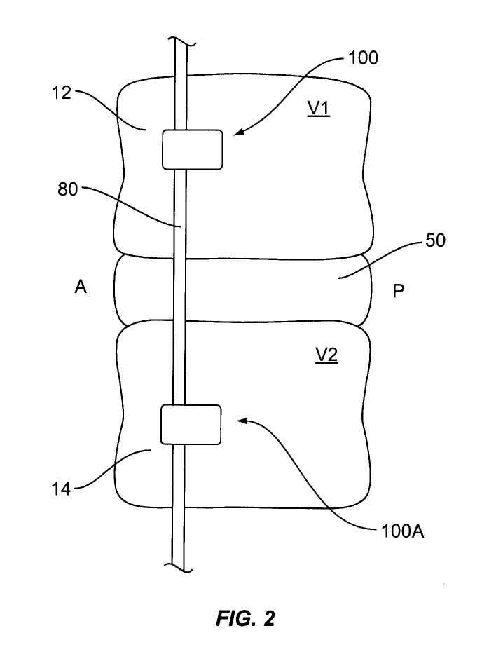

FIG. 2 shows a schematic, side view of the vertebral bodies V1 and V2 of FIG. 1 with an anchoring system 100. The anchoring system 100 is used to anchor at least a portion of material 80 to a vertebral body V1 or V2. As show in FIG. 2, there is an anchoring system 100 affixed to the anterior lateral side of vertebral body V1 and an anchoring system 100A affixed to the anterior lateral side of vertebral body V2. The anchoring system 100 and/or 100A may be affixed to another location on the vertebral bodies V1 and V2, for example, they may be affixed to the pedicles (not shown) on the posterior section of the vertebral bodies V1 and V2.

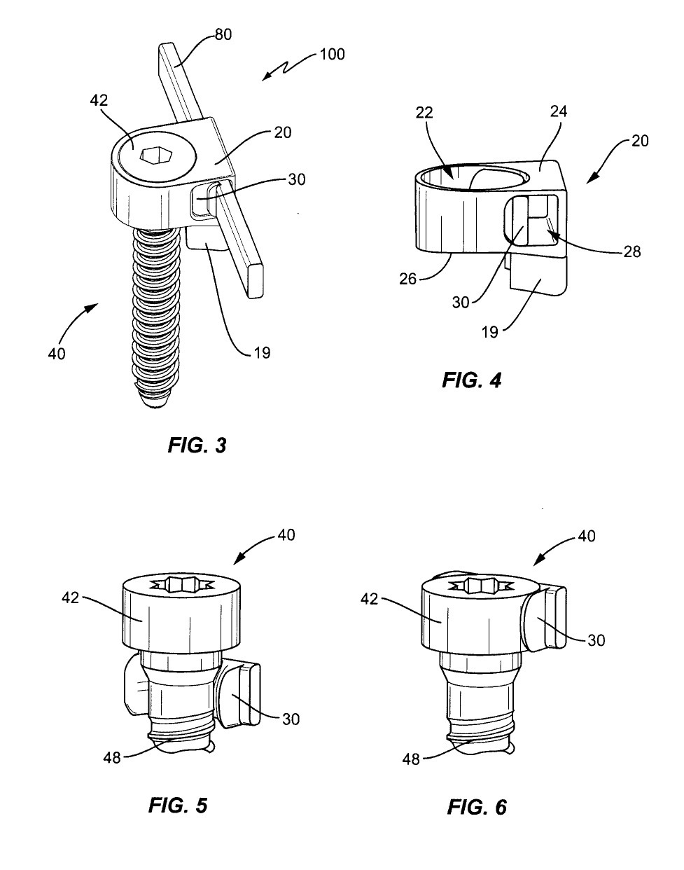

FIG. 3 shows an isometric view of an anchoring system 100. As shown in FIG. 3, the anchoring system 100 comprises a base 20, a fastener 40 and an anchoring mechanism 30. The base 20 is configured to affix to the vertebral body V1 or V2, configured to receive the fastener 40 and configured to receive the at least a portion of material 80. The anchoring mechanism 30 is configured to engage with the fastener 40, configured to engage with the base 20 and configured to anchor the at least a portion of material 80 to the base 20, wherein the mechanism 30 comprises at least one elastic element 30 configured to apply pressure to the at least a portion of material 80 so as to anchor the at least a portion of material 80 to the base 20. Further, as shown in FIG. 3, the underside of the base 20 may have structures 19 such as anchors, keels, spikes, pegs, prongs, or similar structures to help affix base 20 to the vertebral body V1 or V2 and/or help maintain its proper placement on the vertebral body V1 or V2 and/or absorb some of the load on the base 20.

FIG. 4 shows an isometric view of the base 20 and the anchoring mechanism 30 of the anchoring system 100 of FIG. 3. As shown in FIG. 4, the base 20 comprises at least one hole 22 for receiving the fastener 40, which is configured for affixing the base 20 to the vertebral body V1 or V2. The base 20 further comprise slots 28 for accommodating at least a portion of the at least a portion of material 80. Also, slots 28 may accommodate a portion of the anchoring mechanism 30. As shown in FIG. 3, anchoring mechanism 30 has been placed within base 20 and positioned so as to allow for placement of the at least a portion of material 80.

FIG. 5 shows an isometric view of the anchoring mechanism 30 of the anchoring system 100 of FIG. 3 in cooperation with a fastener 40. As shown in FIG. 5, the fastener 40 has a fastener head 42 and a fastener shank 48. Fastener shank 48 is configured for affixing to the vertebral body V1 or V2 and the fastener 40 is manipulated by using the fastener head 42. FIG. 6 shows another isometric view of the anchoring mechanism 30 of the anchoring system 100 of FIG. 3 in cooperation with a fastener 40. In particular, FIG. 6 shows fastener 40 in its fully-inserted position. In its fully-inserted position, fastener head 42 abuts anchoring mechanism 30, as shown in FIG. 6. In this way, fastener 40 may impart a force against the anchoring mechanism 30 so that the anchoring mechanism 30 applies pressure to the at least a portion of material 80 so as to anchor the at least a portion of material 80 to the base 20.

As shown in FIGS. 3-6, the anchoring mechanism 30 comprises an elastic element 30 that has elasticity derived from the physical characteristics of its material. Note, however, that the elastic element 30 may be, for example, a coil spring. As shown, such elastic element 30 may include but not be limited to, any one or any combination of a polymer or other biocompatible material. Further, while the elastic components are elastic, they may be non-rigid. For example, suitable materials for the elastic element 30 may, for example, include but not be limited to, latex, rubber, silicone, polyurethane, silicone-polyurethane copolymers, and/or polyolefin rubbers.

In operation of the anchoring system depicted in FIGS. 3-6, the anchoring mechanism 30 is placed within base 20 and positioned so as to allow for placement of the at least a portion of material 80, as shown in FIG. 4. The at least a portion of material 80 is then placed within the base 20. The aforementioned steps may occur before or after the base 20 is placed on a vertebral body, for example, V1 or V2, whereby structures 19 may help maintain proper placement of the base 20 on the vertebral body and/or absorb some of the load on the base 20. Thereafter, the fastener 40 is placed within hole 22, entering the hole 22 by way of surface 24, and used to affix the base 20 in the desired location on the vertebral body.

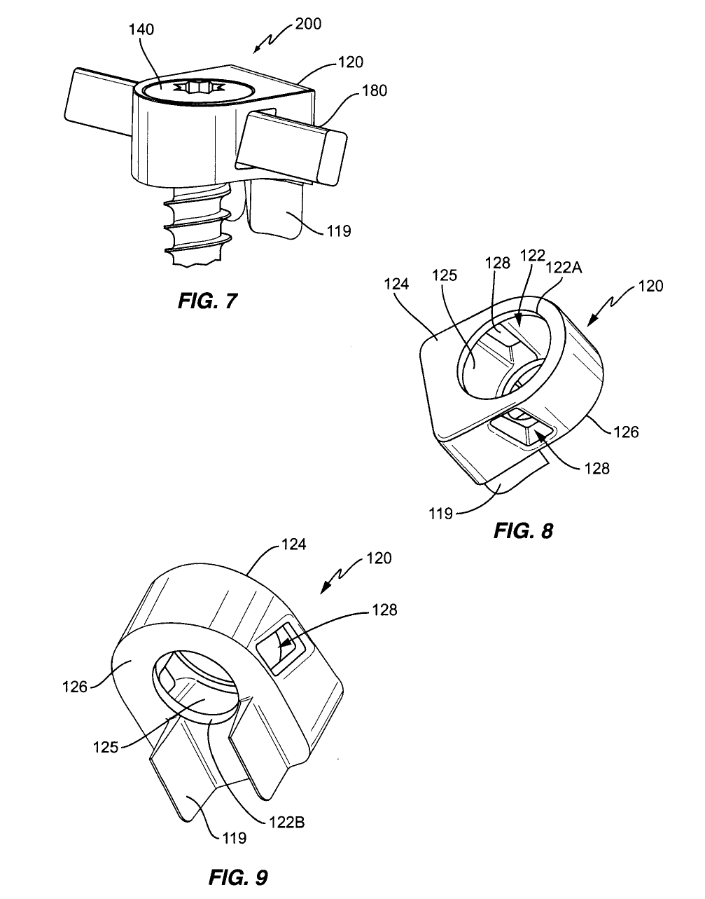

FIG. 7 shows an isometric view of an anchoring system 200. As shown in FIG. 7, the anchoring system 200 comprises a base 120 and a fastener 140. The base 120 is configured to affix to the vertebral body V1 or V2, configured to receive the fastener 140 and configured to receive the at least a portion of material 180 so as to apply pressure to the at least a portion of material 180 so as to anchor the at least a portion of material 180 to the base 120. Further, as shown in FIG. 7, the underside of the base 120 may have structures 119 such as anchors, keels, spikes, pegs, prongs, or similar structures to help affix the base 120 to the vertebral body V1 or V2 and/or help maintain its proper placement on the vertebral body V1 or V2 and/or absorb some of the load on the base 120.

FIG. 8 shows a top isometric view of the base 120 of the anchoring system 100 of FIG. 7. As shown in FIG. 8, the base 120 comprises at least one hole 122 for receiving a fastener 140, which is configured for affixing the base 120 to the vertebral body V1 or V2. The base 120 further comprise slots 128 for accommodating at least a portion of the at least a portion of material 180. As shown in FIG. 8, the base 120 has a top surface 124 and a bottom surface 126. As shown in FIG. 8, between the top surface 124 and the bottom surface 126, the base 120 comprises a surface 125 configured to engage a first surface 184 of the at least a portion of material 180. As shown in FIG. 8, the shape of surface 125 is frustoconical. The fastener 140 is configured to engage a second surface 182 of the at least a portion of material 180, wherein the first surface 184 and second surface 182 of the at least a portion of material 180 face substantially opposite directions. The top surface 124 is an outer surface that receives the fastener 140 and the bottom surface 126 is an inner surface that engages the vertebral body V1 or V2. Note that while, as shown, surface 124 receives the fastener 140, surface 125 contacts the fastener 140, but surface 124 does not contact the fastener. As shown, the structures 119 are attached to the bottom surface 126.

The terms “generally” (or “general”) or “substantially” (or “substantial”) as used herein may be applied to modify any quantitative representation which could permissibly vary without resulting in a change in the basic function to which it is related. For example, while the first surface 184 and second surface 182 of the at least a portion of material 180 face substantially opposite directions, the surfaces need only face directions that allow the base 120 and the fastener 140 to compress the at least a portion of material 180 so as to anchor the at least a portion of material 180 to the base 120.

FIG. 9 shows a bottom isometric view of the base 120 of the anchoring system 200 of FIG. 7. As shown in FIG. 9, the bottom surface 126 of the base 120 has two structures 119. Further, as shown in FIG. 9, the hole 122 for receiving fastener 140 has a larger opening 122A at the top surface 124 than its opening 122B at the bottom surface 126 of the base 120. As shown in FIG. 9, as surface 125 extends between the openings 122A and 122B, the surface 125 is angled with respect to a longitudinal axis of the fastener 140.

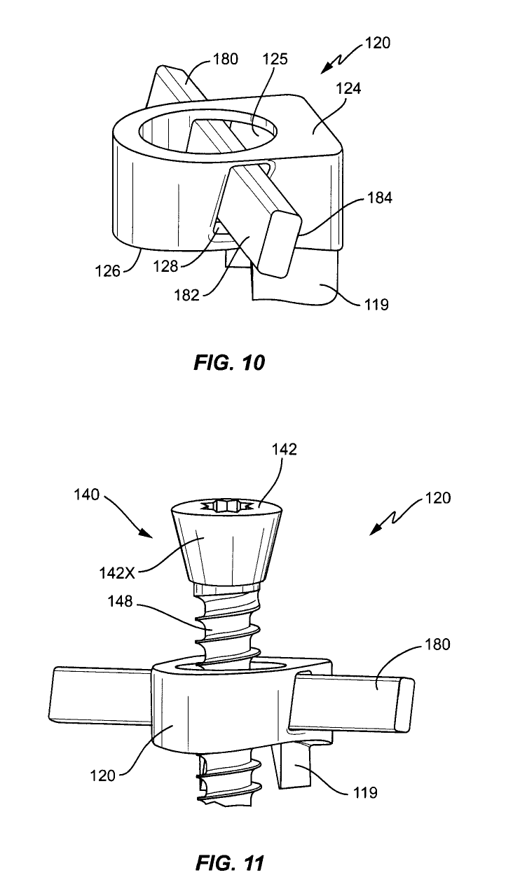

FIG. 10 shows an isometric view of the base 120 of the anchoring system 200 of FIG. 7 in cooperation with the at least a portion of material 180. FIG. 10 shows that surface 125 extends between the top 124 and bottom 126 surfaces of the hole 122 and that the surface 125 is angled with respect to a longitudinal axis of the fastener 140. As shown in FIG. 10, surface 125 is configured to engage the first surface 184 of the at least a portion of material 180 and the fastener 140 is configured to engage a second surface 182 of the at least a portion of material 180, wherein the first surface 184 and second surface 182 of the at least a portion of material 180 face substantially opposite directions.

FIG. 11 shows an isometric view of the base 120 and fastener 140 of the anchoring system 200 of FIG. 7 in cooperation with the at least a portion of material 180. As shown in FIG. 11, the fastener 140 has a fastener head 142 and a fastener shank 148. Fastener shank 148 is configured for penetration and affixation to the vertebral body V1 or V2 and the fastener 140 is manipulated by using the fastener head 142. Further, as shown, the fastener head 142 has a surface 142X that is angled to cooperate with the at least a portion of material 180 and surface 125 of the base 120.

In operation of the anchoring system depicted in FIGS. 7-11, the at least a portion of material 180 is placed within base 120, as shown in FIG. 10. This step may occur before or after the base 120 is placed on a vertebral body, for example, V1 or V2, whereby structures 119 may maintain proper placement of the base 120 on the vertebral body and/or absorb some of the load on the base 120. Thereafter, as shown in FIG. 11, the fastener 140 is placed within hole 122, entering the hole 122 by way of surface 124, and used to affix the base 120 in the desired location on the vertebral body, thereby anchoring the at least a portion of material 180 to the base 120.

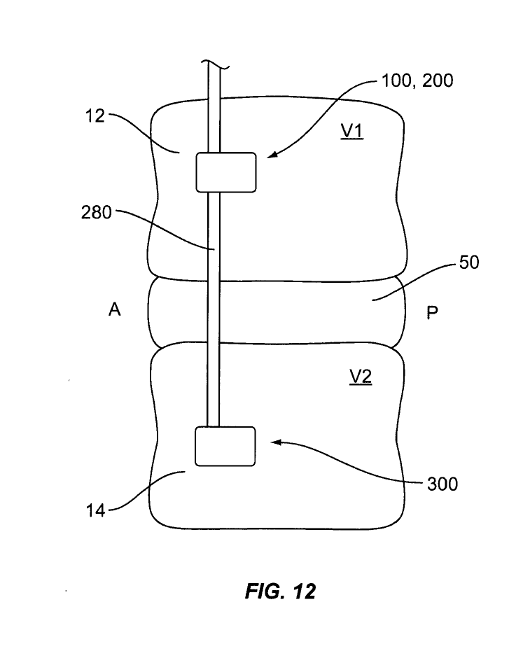

FIG. 12 shows a schematic, side view of the vertebral bodies V1 and V2A with an anchoring system 300 associated with vertebral body V2A. The anchoring system 300 is used to anchor at least a portion of material 280 to vertebral body V2A. As shown, there is an anchoring system 100 or 200 affixed to the anterior lateral side of vertebral body V1 and an anchoring system 300 affixed to the anterior lateral side of vertebral body V2A. The anchoring system 100, 200 or 300 may be affixed to another location of the vertebral bodies V1 and V2A, for example, they may be affixed to the pedicles (not shown) on the posterior section of the vertebral bodies V1 and V2A. As shown, the at least a portion of material 280 terminates at anchoring system 300 on vertebral body V2A.

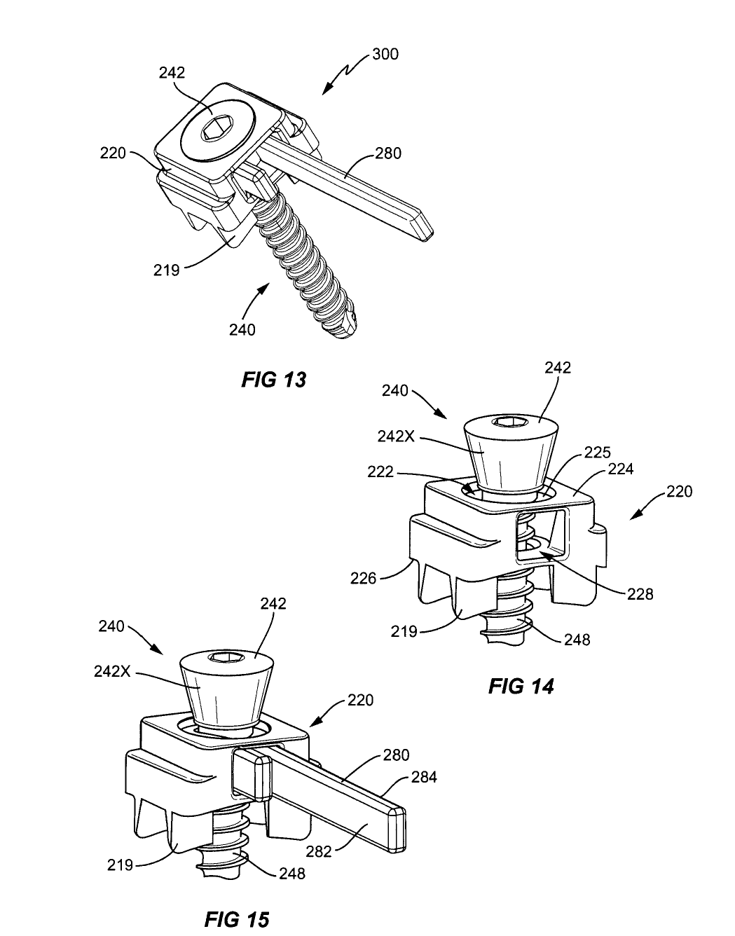

FIG. 13 shows an isometric view of an anchoring system 300. As shown in FIG. 13, the anchoring system 300 comprises a base 220 and a fastener 240. The base 220 is configured to affix to the vertebral body V2A, configured to receive the fastener 240 and configured to receive the at least a portion of material 280 so as to apply pressure to the at least a portion of material 280 so as to anchor the at least a portion of material 280 to the base 220. Further, as shown in FIG. 13, the underside of the base 220 may have structures 219 such as anchors, keels, spikes, pegs, prongs, or similar structures to help affix the base 220 to the vertebral body V2A and/or help maintain its proper placement on the vertebral body V2A and/or absorb some of the load on the base 220.

FIG. 14 shows a top isometric view of the base 220 of the anchoring system 300 of FIG. 13. As shown in FIG. 14, the base 220 comprises at least one hole 222 for receiving a fastener 240, which is configured for affixing the base 220 to the vertebral body V2A. The base 220 further comprise slots 228 for accommodating at least a portion of the at least a portion of material 280. As shown in FIG. 14, the base 220 has a top surface 224 and a bottom surface 226. As shown in FIG. 14, between the top surface 224 and the bottom surface 226, the base 220 comprises a surface 225 configured to engage a first surface 284 of the at least a portion of material 280. The fastener 240 is configured to engage a second surface 282 of the at least a portion of material 280, wherein the first surface 284 and second surface 282 of the at least a portion of material 280 face substantially opposite directions. Specifically, the head 242 of fastener 240 has a surface 242X that engages the second surface 282 of the at least a portion of material 280. The top surface 224 of the base 240 is an outer surface that receives the fastener 240 and the bottom surface 226 is an inner surface that engages the vertebral body V2A. As shown, the structures 219 are attached to the bottom surface 226. Further, note that base 240 has four structures 219.

FIG. 15 shows an isometric view of the base 220 of the anchoring system 300 of FIG. 13 in cooperation with the at least a portion of material 280. As shown in FIG. 15, the fastener 240 has not been fully inserted in the base 240. That is, as shown, surface 242X of fastener head 242 is not engaged with the second surface 282 of the at least a portion of material 280, but is in alignment to do so when it is fully inserted in the base 240. Further, as shown in FIGS. 14 and 15, surface 242X extends between the top of head 242 and the shank 248, and surface 242X is angled with respect to a longitudinal axis of the fastener 240. As shown, surface 242X is configured to engage the second surface 282 of the at least a portion of material 280.

In operation of the anchoring system depicted in FIGS. 13-15, the at least a portion of material 280 is placed within base 220, as shown in FIG. 15. This step may occur before or after the base 220 is placed on a vertebral body, for example, V2, whereby structures 219 may help maintain proper placement of the base 220 on the vertebral body and/or absorb some of the load on the base 220. Thereafter, as shown in FIG. 15, the fastener 240 is placed within hole 222, entering the hole 222 by way of surface 224, and used to affix the base 220 in the desired placement on the vertebral body.

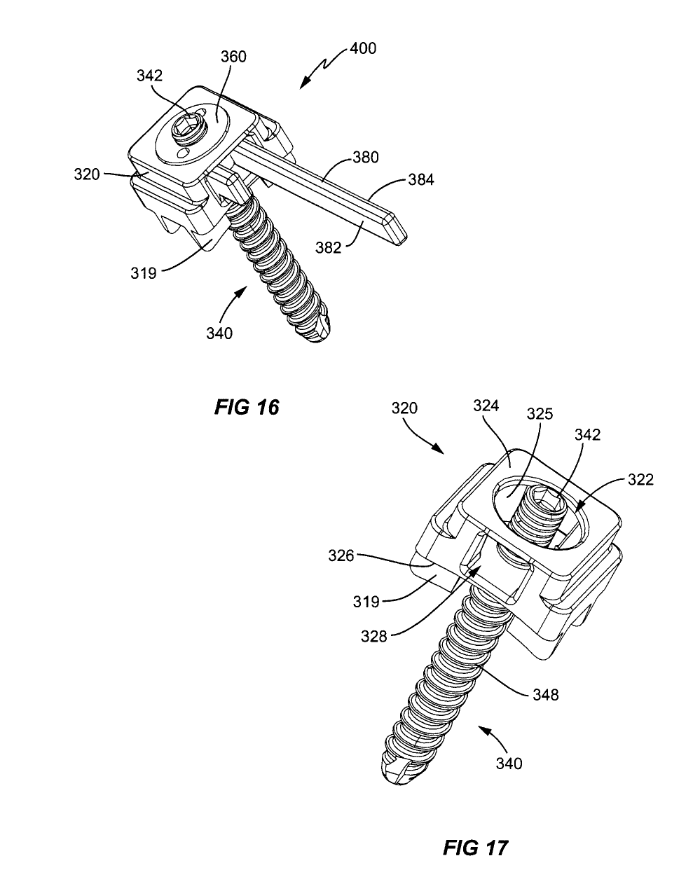

FIG. 16 shows an isometric view of an anchoring system 400. As shown in FIG. 16, the anchoring system 400 comprises a base 320, a fastener 340 and a locking component 360. The base 320 is configured to affix to the vertebral body V2A, configured to receive the fastener 340 and the locking component 360, and configured to receive the at least a portion of material 380 so as to apply pressure to the at least a portion of material 380 so as to anchor the at least a portion of material 380 to the base 320. As shown in FIG. 16, the fastener 340 is a screw and the locking component 360 is a nut. Further, as shown in FIG. 16, the underside of the base 320 may have structures 319 such as anchors, keels, spikes, pegs, prongs, or similar structures to help affix the base 320 to the vertebral body V2A and/or help maintain its proper placement on the vertebral body V2A and/or absorb some of the load on the base 320.

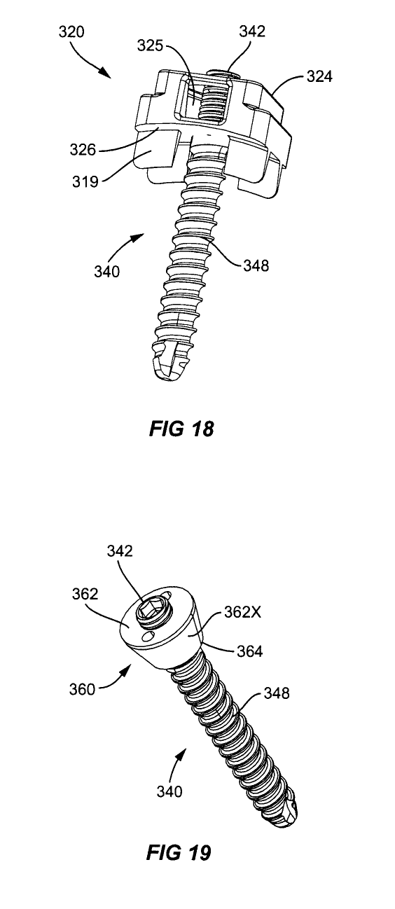

FIG. 17 shows a top isometric view of the base 320 of the anchoring system 400 of FIG. 16, and FIG. 18 shows a bottom isometric view of the base 320 of the anchoring system 400 of FIG. 16. As shown, the base 320 comprises at least one hole 322 for receiving a fastener 340, which is configured for affixing the base 320 to the vertebral body V2A. The base 320 further comprise slots 328 for accommodating at least a portion of the at least a portion of material 380. As shown, the base 320 has a top surface 324 and a bottom surface 326. As shown, between the top surface 324 and the bottom surface 326, the base 320 comprises a surface 325 configured to engage a first surface 384 of the at least a portion of material 380. The nut 360 is configured to engage a second surface 382 of the at least a portion of material 380, wherein the first surface 384 and second surface 382 of the at least a portion of material 380 face substantially opposite directions. Specifically, the surface 362X of nut 360 engages the second surface 382 of the at least a portion of material 380. The top surface 324 of the base 340 is an outer surface that receives the fastener 340 and the bottom surface 326 is an inner surface that engages the vertebral body V2A. As shown, the structures 319 are attached to the bottom surface 326. Further, note that base 340 has four structures 319.

FIG. 19 shows an isometric view of the fastener 340 and locking element 360 of the anchoring system 400 of FIG. 16. As shown, the fastener 340 is a screw and the locking element 360 is a nut. Further, FIG. 19 shows the surface 362X of nut 360, which engages the second surface 382 of the at least a portion of material 380. As shown in FIGS. 17, 18 and 19, the head 342 of fastener 340 (or proximal end) has a smaller diameter than the shank 348 of the fastener 340 (or distal end).

In operation of the anchoring system depicted in FIGS. 16-19, the fastener 340 is placed into the vertebral body, for example, V2A. If the fastener 340 is a screw, for example, then the screw 340 is screwed into the vertebral body such that the shank 348 engages the vertebral body. Then, the at least a portion of material 380 is placed within base 320. This step may occur before or after the base 320 is placed on the vertebral body, whereby structures 319 may help maintain proper placement of the base 320 on the vertebral body and/or absorb some of the load on the base 320. Thereafter, and after the base 320 is placed on the vertebral body over the head 342, the locking element 360 or nut is placed on the head 342 so as to secure the fastener 340 to the base 320 and so as to anchor the at least a portion of material 380 to the base 320.

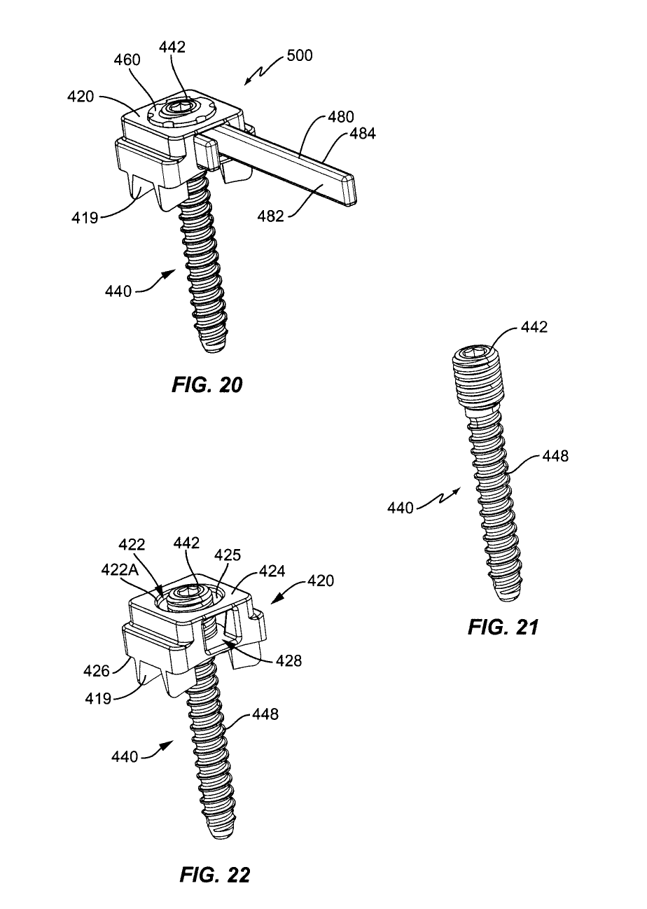

FIG. 20 shows an isometric view of an anchoring system 500. As shown in FIG. 20, the anchoring system 500 comprises a base 420, a fastener 440 and a locking component 460. The base 420 is configured to affix to the vertebral body V2A, configured to receive the fastener 440 and the locking component 460, and configured to receive the at least a portion of material 480 so as to apply pressure to the at least a portion of material 480 so as to anchor the at least a portion of material 480 to the base 420. As shown in FIG. 20, the fastener 440 is a screw and the locking component 460 is a nut. Further, as shown in FIG. 20, the underside of the base 420 may have structures 419 such as anchors, keels, spikes, pegs, prongs, or similar structures to help affix the base 420 to the vertebral body V2A and/or help maintain its proper placement on the vertebral body V2A and/or absorb some of the load on the base 420.

FIG. 21 shows an isometric view of the fastener 440 of the anchoring system 500 of FIG. 20. As shown in FIG. 21, the head 442 of fastener 440 (or proximal end) has a larger diameter than the shank 448 of the fastener 440 (or distal end).

FIG. 22 shows a top isometric view of the base 420 of the anchoring system 500 of FIG. 20 in cooperation with the fastener 440. As shown, the base 420 comprises at least one hole 422 for receiving a fastener 440, which is configured for affixing the base 420 to the vertebral body V2A. The base 420 further comprise slots 428 for accommodating at least a portion of the at least a portion of material 480. As shown, the base 420 has a top surface 424 and a bottom surface 426. As shown, between the top surface 424 and the bottom surface 426, the base 420 comprises a surface 425 configured to engage a first surface 484 of the at least a portion of material 480. The nut 460 is configured to engage a second surface 482 of the at least a portion of material 480, wherein the first surface 484 and second surface 482 of the at least a portion of material 480 face substantially opposite directions. Specifically, the surface 462X of nut 460 engages the second surface 482 of the at least a portion of material 480. The top surface 424 of the base 440 is an outer surface that receives the fastener 440 and the bottom surface 426 is an inner surface that engages the vertebral body V2A. As shown, the structures 419 are attached to the bottom surface 426. Further, note that base 440 has four structures 419.

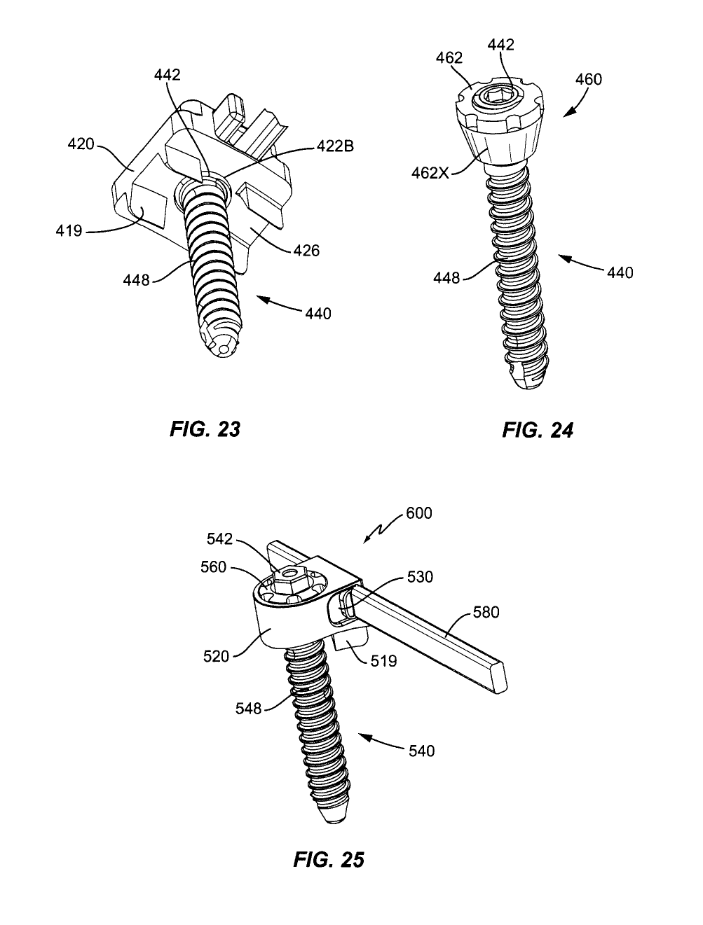

FIG. 23 shows a bottom isometric view of the base 420 and fastener 440 of the anchoring system 500 of FIG. 20. As shown, the fastener 440 is a screw in which the head 442 of fastener 440 (or proximal end) has a larger diameter than the shank 448 of the fastener 440 (or distal end). Further, as shown in FIGS. 22 and 23, hole 422B on the bottom surface 426 of the base 420 is smaller than hole 422A on the top surface 424 of the base 420. Consequently, the head 442 of fastener 440 may be placed through the hole 422A on the top surface 424 of the base 420, but not through the bottom surface 426 of the base 420.

FIG. 24 shows an isometric view of the fastener 440 and locking element 460 of the anchoring system 500 of FIG. 20. As shown, the fastener 440 is a screw and the locking element 460 is a nut. Further, FIG. 24 shows the surface 462X of nut 460. Surface 462X engages the second surface 482 of the at least a portion of material 480.

In operation of the anchoring system depicted in FIGS. 20-24, the base 420 is placed on the vertebral body, for example, V2A, whereby structures 419 may help maintain proper placement of the base 420 on the vertebral body and/or absorb some of the load on the base 420. This step may occur before or after the at least a portion of material 480 is placed within base 420. Thereafter, the fastener 440 is placed through the base 420 and into the vertebral body. If the fastener 440 is a screw, then the screw 440 is screwed into the vertebral body such that the shank 448 engages the vertebral body. Thereafter, the locking element 460 or nut is placed on the head 442 so as to secure the fastener 440 to the base 420 and so as to anchor the at least a portion of material 480 to the base 420.

FIG. 25 shows an isometric view of an anchoring system 600. As shown in FIG. 25, the anchoring system 600 comprises a base 520, a fastener 540, a locking component 560 and an anchoring mechanism 530. The base 520 is configured to affix to the vertebral body V1 or V2, configured to receive the fastener 540, configured to receive the locking component 560 and configured to receive the at least a portion of material 580. The anchoring mechanism 530 is configured to engage with the locking component 560, configured to engage with the base 520 and configured to anchor the at least a portion of material 580, wherein the mechanism 530 comprises at least one elastic element 530 configured to apply pressure to the at least a portion of material 580 so as to anchor the at least a portion of material 580 to the base 520. Further, as shown in FIG. 25, the underside of the base 520 may have structures 519 such as anchors, keels, spikes, pegs, prongs, or similar structures to help affix the base 520 to the vertebral body V1 or V2 and/or help maintain its proper placement on the vertebral body V1 or V2 and/or absorb some of the load on the base 520.

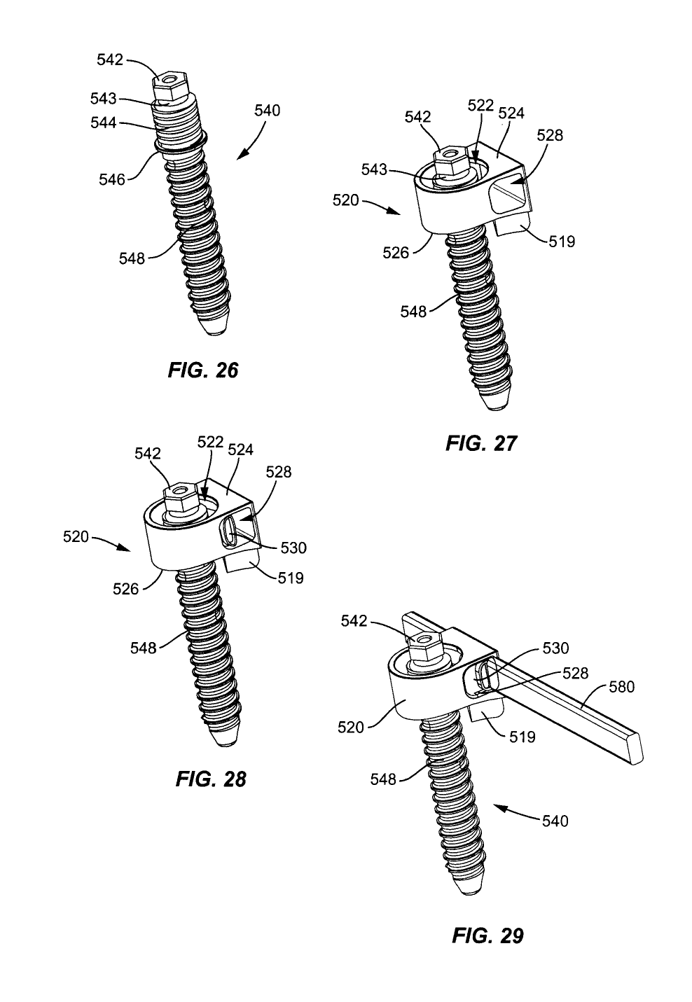

FIG. 26 shows an isometric view of the fastener 540 of the anchoring system 600 of FIG. 24. As shown in FIG. 26, the fastener is a screw and the fastener 540 has a head 542, a proximal shank 544, a shoulder 546, and a distal shank 548. A user manipulates the screw 540 by manipulating the head 542. The locking component 560 engages the proximal shank 544, the distal shank engages the vertebral body V1 or V2 and the shoulder 546 is situated between the proximal shank 544 and the distal shank 548, is configured to engage the base 520 and is configured to limit penetration of the screw 540 into the vertebral body and/or to help alleviate the axial forces that may be distributed to the fastener/bone interface when the fastener 540 is tightened.

FIG. 27 shows an isometric view of the base 520 and the fastener 540 of the anchoring system 600 of FIG. 25. As shown in FIG. 27, the base 520 comprises at least one hole 522 for receiving the fastener 540, which is configured for affixing the base 520 to the vertebral body V1 or V2. The base 520 further comprise slots 528 for accommodating at least a portion of the at least a portion of material 580. Also, slots 528 may accommodate a portion of the anchoring mechanism 530.

FIG. 28 shows an isometric view of the base 520 and the anchoring mechanism 530 of the anchoring system 600 of FIG. 25 in cooperation with the fastener 540. As shown in FIG. 28, the anchoring mechanism 530 has been placed within base 520 and positioned so as to allow for placement of the at least a portion of material 580.

FIG. 29 shows an isometric view of the base 520 and the anchoring mechanism 530 of the anchoring system 600 of FIG. 25 in cooperation with the fastener 540 and the at least a portion of material 580. As shown in FIG. 29, the anchoring mechanism 530 has been placed within base 520 and positioned so as to allow for placement of the locking component 560.

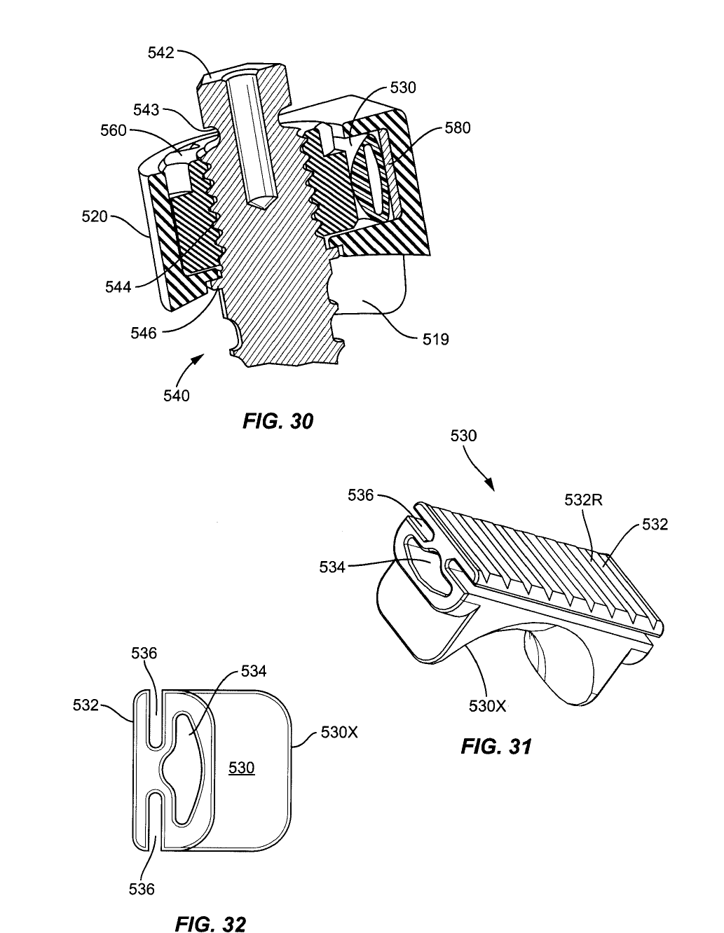

FIG. 30 shows a cross-sectional view of the base 520 and the anchoring mechanism 530 of the anchoring system 600 of FIG. 25 in cooperation with the fastener 540, the at least a portion of material 580 and the locking component 560. As shown in FIG. 30, a user may manipulate the screw 540 by manipulating the head 542. Also, note that head 542 may be a break-off head, i.e., a head that once used for manipulating can be broken off so as to remove the head or portion thereof that has served its purpose. For example, the head 542 may break off at or near neck 543 of screw 540. The locking component 560 engages the proximal shank 544, the distal shank engages the vertebral body V1 or V2 and the shoulder 546 is configured to limit penetration of the screw 540 into the vertebral body and is configured to engage the base 520 and/or to help alleviate the axial forces that may be distributed to the fastener/bone interface when the fastener 540 is tightened. Also, once the locking component 560 is in its fully-inserted position as shown in FIG. 30, the locking component 560 imparts a force radially-outward and upon the anchoring mechanism 530 so as to apply pressure to the at least a portion of material 580 so as to anchor the at least a portion of material 580 to the base 520.

The elastic element 530 may include but not be limited to, any one or any combination of a polymer or other biocompatible material. Further, while the elastic components are elastic, they may be non-rigid. For example, suitable materials for the elastic element 530 may, for example, include but not be limited to, latex, rubber, silicone, polyurethane, silicone-polyurethane copolymers, and/or polyolefin rubbers.

In operation of the anchoring system 600 depicted in FIGS. 25-30, the anchoring mechanism 530 is placed within base 20 and positioned so as to allow for placement of the at least a portion of material 580, as shown in FIG. 28. The at least a portion of material 580 is then placed within the base 520, as shown in FIG. 29. As shown, the base 520 should be placed over the fastener 540, and the fastener should be inserted into the vertebral body before or after placement of the base 520 over the fastener 540. Once the base is affixed to the vertebral body, for example, V1 or V2, structures 519 may help maintain proper placement of the base 520 on the vertebral body and/or absorb some of the load on the base 520. Thereafter, the locking component 560 is placed in position so as to engage the proximal shank 544 of the fastener 540 so as to anchor the at least a portion of material 580 to the base 520.

FIG. 31 shows an isometric view of an anchoring mechanism 530 of the anchoring system 600 of FIG. 25. As shown in FIG. 31, the anchoring mechanism 530 is an elastic element 530. Elastic element 530 has a first surface 530X that is configured for engaging the locking component 560 and a second surface 532 that is configured for engaging the at least a portion of material 580. As shown in the embodiment of FIG. 31, for example, surfaces 532 and 530X are rigid and substantially do not deform during use. Elastic element 530 comprises at least one pocket of air 534. Also, as shown, the elastic element 530 of FIG. 31 comprises additional areas of air 536 (shown as recesses in FIG. 31). As shown in the embodiment of FIG. 31, for example, areas 534 and 536 work together to provide movement of surface 532 toward surface 530X while the elastic element 530 experiences elastic deformation and, in some cases, experiences plastic deformation. When in its fully-inserted position on the base 520, the areas of air 536 are enclosed and may act in a similar fashion to the pocket of air 534. Areas of air (or “open” space”) such as areas 534 and 536 help compress the elastic element 530 and apply pressure to the at least a portion of material 580 in a more uniform manner, thereby help maintaining the at least a portion of material 580 affixed to the base 520. As shown in the embodiment of FIG. 31, for example, the areas 534 and 536 may allow for compression of the elastic element 530 and apply pressure to the at least a portion of material 580 even if the elastic element 530 experiences some creep or other deformation and/or if at least a portion of material 580 experiences the same under load and/or after implantation. In addition, the second surface 532 of the elastic element 530 of FIG. 31 has a plurality of recesses 532R that help the second surface 532 engage the at least a portion of material 580.

FIG. 32 shows a top view of the anchoring mechanism 530 of FIG. 31. FIG. 32 shows another view of areas 534 and 536 and surfaces 530X and 532, and how each is oriented with respect to each other.

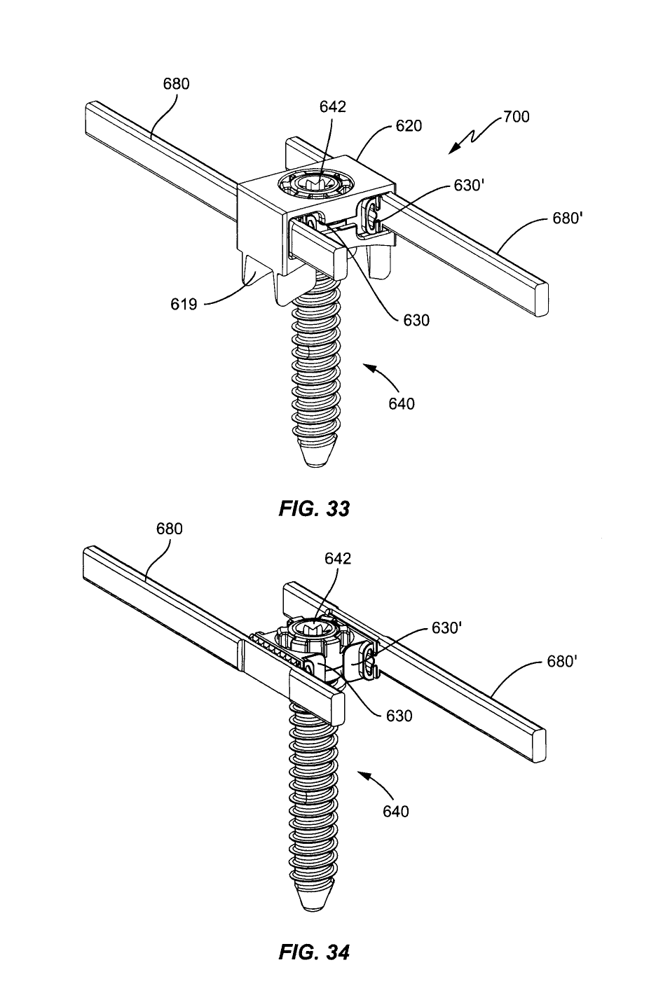

FIG. 33 shows an isometric view of an anchoring system 700 that may utilize, for example and as shown, anchoring mechanisms 630 and 630′ similar to that of FIGS. 31 and 32. As shown in FIG. 33, the anchoring system 700 comprises a base 620, a fastener 640 and an anchoring mechanism 630. The base 620 is configured to affix to the vertebral body V1 or V2, configured to receive the fastener 640 and configured to receive the at least a portion of material 680 and the at least a portion of material 680′. The anchoring mechanism 530 is configured to engage with the base 620 and configured to anchor the at least a portion of material 680, wherein the mechanism 630 comprises at least one elastic element 630 configured to apply pressure to the at least a portion of material 680 so as to anchor the at least a portion of material 680 to the base 620. The anchoring mechanism 630′ is configured to engage with the base 620 and configured to anchor the at least a portion of material 680′, wherein the mechanism 630′ comprises at least one elastic element 630′ configured to apply pressure to the at least a portion of material 680′ so as to anchor the at least a portion of material 680′ to the base 620. Further, as shown in FIG. 33, the underside of the base 620 may have structures 619 such as anchors, keels, spikes, pegs, prongs, or similar structures to help affix the base 620 to the vertebral body V1 or V2 and/or help maintain its proper placement on the vertebral body V1 or V2 and/or absorb some of the load on the base 620.

FIG. 34 shows an isometric view of the anchoring system 700 of FIG. 33 without the base 620. FIG. 34 shows another view of anchoring mechanisms 630 and 630′ as well as the head 642 of fastener 640.

With any of the embodiments described above, the at least a portion of material is non-rigid, and may be flexible. Further, as stated, the at least a portion of material may be a tether or part of a tether that connects an anchoring system 100, 200, 300, 400, 500, 600 and/or 700 to something else, for example, to another anchoring system on an adjacent vertebral body. Examples of such systems are shown in FIGS. 2 and 12. The at least a portion of material may be any one or combination of a cloth, metal, solid polymer, fabric, mesh, or other biocompatible material. Some polymer materials may include, but not be limited to, any one or combination of polyethylene, polyester, polyvinyl, polyvinyl alcohol, polyacrylonitrile, polyamide, polytetrafluoroethylene, poly-paraphenylene and terephthalamide. Further, the at least a portion of material 80, 180, 280, 380, 480, 580, 680 and/or 680′ may be made of a suture wire of polyetheretherketone (“PEEK”), polyester or polyethylene. In addition, the at least a portion of material 80, 180, 280, 380, 480, 580, 680 and/or 680′ may be elastic, woven, knitted, braided or flexible. Some woven, knitted or braided materials may, for example, include nylon, Dacron®, and/or woven fibers or filaments of polyester, polyethelene, polypropylene, PEEK, polytetrafluoroethylene (“PTFE”), and/or woven PEEK. Some elastic materials may, for example, include latex, rubber, silicone, polyurethane, silicone-polyurethane copolymers, and/or polyolefin rubbers. Other suitable materials may, for example, include Gore-Tex®, Kevlar®, Spectra, polyether, polycarbonate urethane, shape memory material with pseudo elastic or superelastic characteristics, metals, metal alloys, and polymers, braided polymers, synthetic resorbable materials such as polyactide, polygycolide, polyorthoester, calcium phosphate, and/or glass, nonresorbable polyethylene, cellulose, materials that are potentially absorbable, and/or materials that are used in making artificial ligaments. Further, suitable materials should be non-biodegradable and non-resorbable. In addition to woven, braided, or knitted structures, the at least a portion of material 80, 180, 280, 380, 480, 580, 680 and/or 680′ also may be composed of non-woven structures such as non-woven mesh or chained structures.

Further, note that the various components of the anchoring systems 100, 200, 300, 400, 500, 600 and/or 700 may be made of a variety of materials and any combination thereof. Suitable materials for any component other than the at least a portion of material 80, 180, 280, 380, 480, 580, 680 and/or 680′ include, but are not limited to, any one or any combination of a metal, metal alloy (for example, Titanium alloys or Nitinol) polymer (for example, strong plastic or polymer material with low creep, such as PEEK), ceramic, or other biocompatible material.

Except for the elastic elements 30, 530, 630 and 630′, the components should be rigid. Also, note that, as shown, while elastic elements 30, 530, 630 and 630′ comprise elastic portions, they may comprise portions that are rigid and/or non-elastic. Accordingly, while at least a portion of the elastic elements 30, 530, 630 and 630′ are elastic, they may be non-rigid. Further, suitable materials for at least a portion of the elastic elements 30, 530, 630 and 630′ may, for example, include but not be limited to, latex, rubber, silicone, polyurethane, silicone-polyurethane copolymers, and/or polyolefin rubbers. In addition, the areas 534 and 536 may be filled or partially filled with, for example, a highly elastic material that may be used for adjusting the spring properties of elastic elements 30, 530, 630 and 630′.

Further, as an example with respect to the elastic element 530 of FIG. 31, after the locking mechanism (for example, 560) is fully inserted, total deformation of the elastic element 530 may vary from approximately 0.1 mm. to 0.5 mm. As a result, the elastic element 530 may be designed with up to 0.4 mm of plastic deformation to provide a stable spring load caused by 0.1 mm elastic deformation. Such an embodiment will accommodate an anchoring system where the size of the gap between two holding surfaces, before the at least a portion of material (for example, 580) is in place, is approximately 0.1+/−0.1 mm. and the thickness of the at least a portion of material in a fully compressed state is approximately 0.3-0.5 mm. Note that the distances supplied in this paragraph are used solely to help illustrate one example.

All adjustments and alternatives described above are intended to be included within the scope of the invention, as defined exclusively in the following claims. Those skilled in the art also should realize that such modifications and equivalent constructions or methods do not depart from the spirit and scope of the present disclosure, and that they may make various changes, substitutions, and alterations herein without departing from the spirit and scope of the present disclosure. For example, some fasteners disclosed herein may be interchanged with other fasteners. One such example is that fastener 240 may be used to replace fastener 340 and locking component 360 of anchoring system 400 and, similarly, fastener 340 and locking component 360 may be used to replace fastener 240 of anchoring system 300. In addition, although the anchoring systems above are described as being configured to affix at least a portion of material to a vertebral body, they also may affix at least a portion of material to any bone. Similarly, although the anchoring systems are described as being configured to affix to an anterior and/or anterior lateral surface of a vertebral body, they also may affix to a posterior surface of a vertebral body (such as a pedicle), a lateral surface of a vertebral body or any plurality or combination of such surfaces. Further, although the fasteners of the anchoring systems are shown as screws, the fasteners need not be screws, but other fasteners that accomplish the necessary function of the fasteners.

Furthermore, as used herein, the terms components and elements may be interchanged. It is understood that all spatial references, such as “superior,” “inferior,” “anterior,” “posterior,” “above,” “lower,” “outside,” “inside,” “higher,” “lower,” “outer,” “inner,” “extended,” “reduced,” “shorter,” “longer,” and “perimeter” are for illustrative purposes and can be varied within the scope of the disclosure.

1. A system for anchoring at least a portion of material to a vertebral body, the system comprising:
a base configured to affix to the vertebral body and configured to receive the at least a portion of material;
a fastener configured to affix the base to the vertebral body;
an anchoring mechanism configured to engage with the base and configured to anchor the at least a portion of material, wherein the anchoring mechanism comprises at least one elastic element configured to engage with the fastener and configured to apply pressure to the at least a portion of material so as to anchor the at least a portion of material to the base.

2. The system of claim 1, wherein the at least one elastic element is a spring.

3. The system of claim 2, wherein the spring is a compression spring.

4. The system of claim 1, wherein the elastic element has elasticity derived from the physical characteristics of its material.

5. The system of claim 1, wherein the fastener comprises a first component and a second component, wherein the first component is configured for engaging the vertebral body and the second component is a locking component configured for engaging the first component and the elastic element.

6. The system of claim 5, wherein the first component is a screw and the second component is a nut.

7. The system of claim 1 further comprising the at least a portion of material.

8. The system of claim 7, wherein the at least a portion of material is non-rigid and flexible.

9. The system of claim 7, wherein the at least a portion of material is part of a tether.

10. A system for anchoring at least a portion of material to a vertebral body, the system comprising:
a base configured to affix to the vertebral body and configured to receive the at least a portion of material, and configured to receive a fastener so as to apply pressure to the at least a portion of material so as to anchor the at least a portion of material to the base.

11. The system of claim 10 further comprising the fastener, which is configured to apply pressure to the at least a portion of material so as to anchor the at least a portion of material.

12. The system of claim 11, wherein the base comprises a surface configured to engage a first surface of the at least a portion of material and wherein the fastener is configured to engage a second surface of the at least a portion of material, wherein the first surface and second surface of the at least a portion of material face substantially opposite directions.

13. The system of claim 12 further comprising the at least a portion of material.

14. The system of claim 12, wherein the at least a portion of material is non-rigid.

15. The system of claim 12, wherein the at least a portion of material is flexible.

16. The system of claim 11, wherein the fastener comprises a screw and a nut.

17. The system of claim 11, wherein the base further comprises structures to help affix the base to the vertebral body and maintain proper placement on the vertebral body.

18. A system for anchoring at least a portion of material to a vertebral body, the system comprising:
a base configured to affix to the vertebral body and configured to receive the at least a portion of material;
a fastener comprising a screw and a nut, the fastener configured to affix the base to the vertebral body; and
an anchoring mechanism configured to engage with the base and configured to anchor the at least a portion of material, wherein the anchoring mechanism comprises at least one elastic element configured to engage with the nut and configured to apply pressure to the at least a portion of material so as to anchor the at least a portion of material to the base.

19. The system of claim 16 further comprising the at least a portion of material, wherein the at least a portion of material is flexible.

20. The system of claim 18, wherein the at least one elastic element comprises at least one pocket of air.
